(19) United States
(12) Patent Application Publication
(10) Pub. No.: US 2009/0312686 A1
(43) Pub. Date: Dec. 17, 2009

(54) BLOOD PURIFICATION APPARATUS AND PRIMING METHOD THEREOF

(75) Inventors: Kazuya Sakamoto, Shizuoka (JP); Tomohiro Furuhashi, Shizuoka (JP)

Correspondence Address: DARBY & DARBY P.C., P.O. BOX 770, Church Street Station, New York, NY, 10008-0770, US

(73) Assignee: Nikkiso Co., Ltd., Tokyo (JP)

(21) Appl. No.: 12/480,096

(22) Filed: Jun. 8, 2009

(30) Foreign Application Priority Data
Jun. 16, 2008 (JP) 2008-156604

(51) Int. Cl. A61M 1/14

(52) U.S. Cl. 604/6.11

(57) ABSTRACT

An object of the present invention is to provide a blood purification apparatus and a priming method thereof which can firmly perform the bubble purging smoothly and in a short time during priming operation. According to the blood purification apparatus of the present invention, the tip of the arterial blood circuit can be connected to and communicated with the tip of the venous blood circuit during priming of the blood purification apparatus before dialysis, and the blood purification apparatus can perform a priming solution charging step for supplying and charging the blood circuits with priming solution under a condition in which the tip of the arterial blood circuit and the tip of the venous blood circuit are connected to and communicated with each other; and a priming solution circulating step for forcing the charged priming solution to be flowed and circulated through the blood circuits by successively changing the driving speed of the blood pump after the priming solution charging step.

### CROSS REFERENCE TO PRIOR RELATED APPLICATIONS

The present invention claims priority under 35 U.S.C. §119 to Japanese Patent Application No. 2008-156604 filed on Jun. 16, 2008. The content of the application is incorporated herein by reference in its entirety.

### FIELD OF THE INVENTION

The present invention relates to a blood purification apparatus for extracorporeally circulating the blood of a patient to purify the blood and a priming method for priming the blood purification apparatus.

### DESCRIPTION OF THE INVENTION

In general, it is used in dialysis blood circuits for extracorporeally circulating collected blood of a patient and returning it again into a body of a patient. The blood circuits mainly includes an arterial blood circuit and a venous blood circuit adapted to be connected to a dialyzer (blood purification means) provided e.g. with a hollow fiber membrane. An puncture needle can be mounted on each tip of the arterial blood circuit and the venous blood circuit and thus the extracorporeal circulation of blood can be performed in the dialysis with the puncture needles of the arterial blood circuit and the venous blood circuit being connected to the shunts of a patient, respectively.

A Peristaltic blood circulating pump (simply referred to as a blood pump) is arranged intermediately of the arterial blood circuit and the blood of a patient is adapted to be fed to the dialyzer by driving the blood pump. An arterial drip chamber and a venous drip chamber are arranged intermediately of the arterial blood circuit and the venous blood circuit respectively. The blood is returned again to a body of a patient after purification and removal of bubbles therefrom.

A priming solution (saline) supplying line is connected to the arterial blood circuit at a position upstream side (i.e. arterial needle side) of the blood pump via a “T” joint for supplying the saline during the priming and the return of blood. The dialyzer is structured so that the priming is performed before start of dialysis by supplying the saline and charging the structural elements such as blood circuits and drip chambers connected thereto with the saline and then the saline is replaced with blood remained in the blood circuits after the dialysis and finally the remained blood is returned to a patient. A dialyzer furnished with the priming solution supplying line is disclosed e.g. in Japanese Laid-open Patent Publication No. 93449/2000.

However in the blood purification apparatus (dialyzer), there is a concern that bubbles in the priming solution would remain therein without being discharged to the atmosphere when the priming solution is supplied to the blood circuits and charged therein, and thus it is necessary to continue the supply of the priming solution for a relatively long time in order to remove the remaining bubbles in order to discharge them to the atmosphere.

When the blood circuits are charged with the priming solution, bubbles adhered to inner wall surfaces of the blood circuits cannot be easily entrained by the priming solution and thus tend to remain on the inner wall surface of the blood circuits if the priming solution is flowing at a constant rate. Accordingly, it is necessary to continue supplying priming solution for a relatively long time or to apply operator's manual vibration to the structural elements of the blood purification apparatus while monitoring movement of the bubbles in order to ensure discharge of the bubbles to the atmosphere. Thus, in the blood purification apparatus of the prior art, a large quantity of priming solution is required and a long time is required for the priming of the blood purification apparatus.

It is, therefore, an object of the present invention to provide a blood purification apparatus (dialyzer) and a priming method thereof which can reliably perform the bubble purging smoothly and in a short time during priming operation.

### SUMMARY OF THE INVENTION

For achieving the object of the present invention, there is provided according to the present invention a blood purification apparatus having blood circuits including an arterial blood circuit and a venous blood circuit for extracorporeally circulating blood of a patient from a tip of the arterial blood circuit to a tip of the venous blood circuit; a blood purification means for purifying blood of a patient flowing through the blood circuits interposed between the arterial blood circuit and the venous blood circuit of the blood circuits and having a blood purification membrane and formed with a blood route through which blood of a patient flows and a dialysate route through which dialysate flows respectively through the blood purification membrane; a blood pump arranged in the arterial blood circuit; a dialysate introducing line and a dialysate discharging line respectively connected to an introducing port and a discharging port of the dialysate route of the blood purification means; a blood introducing port formed on the blood purification means and adapted to be connected to the arterial blood circuit for introducing blood into the blood route and a blood discharging port formed on the blood purification means and adapted to be connected to the venous blood circuit for discharging blood from the blood route; and a dialysate introducing port formed on the blood purification means and adapted to be connected to the dialysate introducing line for introducing dialysate into the dialysate route and a dialysate discharging port formed on the blood purification means and adapted to be connected to the dialysate discharging line for discharging dialysate from the dialysate route; the tip of the arterial blood circuit can be connected to and communicated with the tip of the venous blood circuit during priming of the blood purification apparatus before dialysis characterized in that the blood purification apparatus can perform; a priming solution charging step for supplying and charging the blood circuits with priming solution under a condition in which the tip of the arterial blood circuit and the tip of the venous blood circuit are connected to and communicated with each other; and a priming solution circulating step for forcing the charged priming solution to be flowed and circulated through the blood circuits by successively changing the driving speed of the blood pump after the priming solution charging step.

It is preferable that the priming solution circulating step is performed by combining any two or more driving states of the blood pump of a normal rotation state, a reverse rotation state, and a stopped state.

It is preferable that at least two different driving speeds of the blood pump can be set in the normal rotation state and the reverse rotation state.

The blood purification apparatus can further include an arterial drip chamber arranged intermediately of the arterial blood circuit; an arterial overflow line extending from the arterial drip chamber and adapted to be opened and closed to communicate an air layer in the arterial drip chamber with the atmosphere; a venous drip chamber arranged intermediately of the venous blood circuit; a venous overflow line extending from the venous drip chamber and adapted to be opened and closed to communicate an air layer in the venous drip chamber with the atmosphere; and a priming solution supplying line for supplying the priming solution connected to the arterial blood circuit at a junction between the tip of the arterial blood line and the blood pump; the priming solution charging step includes a first charging step for normally rotating the blood pump by a discharging quantity corresponding to a volume of the flow route in the venous blood circuit from the blood discharging port of the blood purification means to the venous drip chamber; a second charging step for discharging the priming solution from the venous overflow line after the stop of the blood pump by opening the venous overflow line and then forcing the priming solution to flow from the priming solution supplying line to the venous drip chamber of the venous blood circuit via the junction of the arterial blood circuit and the connection of the tip of the arterial blood circuit and the tip of the venous blood circuit; a third charging step for discharging the priming solution from the arterial overflow line by closing the venous overflow line and opening the arterial overflow line and then forcing the priming solution from the priming solution supplying line to be flowed to the arterial drip chamber of the arterial blood circuit via the junction of the arterial blood circuit, the connection of the tip of the arterial blood circuit and the tip of the venous blood circuit, and the blood route of the blood purification means; and a fourth charging step for discharging the priming solution from the arterial overflow line by normally rotating the blood pump and then forcing the priming solution to flow from the junction of the arterial blood circuit to the arterial drip chamber of the arterial blood circuit via the blood pump.

In one embodiment, the priming solution circulating step includes a first circulating step for circulating the charged priming solution in the blood circuits by closing the venous overflow line and the priming solution supplying line and then forcing the charged priming solution to be flowed; and a second circulating step for purging air from the priming solution by opening the priming solution supplying line and opening either one of the arterial overflow line and the venous overflow line.

Additionally, the priming solution charging step and the priming solution circulating step are performed by forcing the priming solution to be flowed from the bottom to the top of the blood purification means.

Further, the priming solution charging step and the priming solution circulating step are performed by arranging the blood introducing port at the top of the blood purification means and the blood discharging port at the bottom of the blood purification means.

Also, a bubble detecting means for detecting bubbles in the blood circuits is arranged intermediately of the arterial blood circuit or the venous blood circuit, and that the air purging in the second circulating step is performed with interlocking with the detection of bubbles by the bubble detecting means.

The blood purification apparatus can further have a gas purging step for charging the dialysate route with dialysate by introducing the dialysate into the dialysate route of the blood purification means from the dialysate introducing line and discharging the dialysate from the dialysate discharging line, and that the gas purging step is automatically performed in the priming solution charging step and/or the priming solution circulating step.

According to the present invention there is provided a priming method for priming a blood purification apparatus having blood circuits including an arterial blood circuit and a venous blood circuit for extracorporeally circulating blood of a patient from a tip of the arterial blood circuit to a tip of the venous blood circuit; a blood purification means for purifying blood of a patient flowing through the blood circuits interposed between the arterial blood circuit and the venous blood circuit of the blood circuits and having a blood purification membrane and formed with a blood route through which blood of a patient flows and a dialysate route through which dialysate flows respectively through the blood purification membrane; a blood pump arranged in the arterial blood circuit; a dialysate introducing line and a dialysate discharging line respectively connected to an introducing port and a discharging port of the dialysate route of the blood purification means; a blood introducing port formed on the blood purification means and adapted to be connected to the arterial blood circuit for introducing blood into the blood route and a blood discharging port formed on the blood purification means and adapted to be connected to the venous blood circuit for discharging blood from the blood route; and a dialysate introducing port formed on the blood purification means and adapted to be connected to the dialysate introducing line for introducing dialysate into the dialysate route and a dialysate discharging port formed on the blood purification means and adapted to be connected to the dialysate discharging line for discharging dialysate from the dialysate route; the tip of the arterial blood circuit can be connected to and communicated with the tip of the venous blood circuit during priming of the blood purification apparatus before dialysis, characterized in that the priming method of the blood purification apparatus including a priming solution charging step for supplying and charging the blood circuits with priming solution under a condition in which the tip of the arterial blood circuit and the tip of the venous blood circuit are connected to and communicated with each other; and a priming solution circulating step for forcing the charged priming solution to be flowed and circulated through the blood circuits by successively changing the driving speed of the blood pump after the priming solution charging step.

Also, the priming solution circulating step is performed by combining any two or more driving states of the blood pump of a normal rotation state, a reverse rotation state, and a stopped state.

It is also preferable that at least two different driving speeds of the blood pump can be set in the normal rotation state and the reverse rotation state.

It is preferable that in a priming method for priming a blood purification apparatus, the blood purification apparatus further has an arterial drip chamber arranged intermediately of the arterial blood circuit; an arterial overflow line extending from the arterial drip chamber and adapted to be opened and closed to communicate an air layer in the arterial drip chamber with the atmosphere; a venous drip chamber arranged intermediately of the venous blood circuit; a venous overflow line extending from the venous drip chamber and adapted to be opened and closed to communicate an air layer in the venous drip chamber with the atmosphere; and a priming solution supplying line for supplying the priming solution connected to the arterial blood circuit at a junction between the tip of the arterial blood line and the blood pump; and the priming solution charging step comprises a first charging step for normally rotating the blood pump by a discharging quantity corresponding to a volume of the flow route in the venous blood circuit from the blood discharging port of the blood purification means to the venous drip chamber; a second charging step for discharging the priming solution from the venous overflow line after the stop of the blood pump by opening the venous overflow line and then forcing the priming solution from the priming solution supplying line to be flowed to the venous drip chamber of the venous blood circuit via the junction of the arterial blood circuit and the connection of the tip of the arterial blood circuit and the tip of the venous blood circuit; a third charging step for discharging the priming solution from the arterial overflow line by closing the venous overflow line and opening the arterial overflow line and then forcing the priming solution from the priming solution supplying line to be flowed to the arterial drip chamber of the arterial blood circuit via the junction of the arterial blood circuit, the connection of the tip of the arterial blood circuit and the tip of the venous blood circuit, and the blood route of the blood purification means; and a fourth charging step for discharging the priming solution from the arterial overflow line by normally rotating the blood pump and then forcing the priming solution to be flowed from the junction of the arterial blood circuit to the arterial drip chamber of the arterial blood circuit via the blood pump.

In a priming method for priming a blood purification apparatus, the priming solution circulating step includes a first circulating step for circulating the charged priming solution in the blood circuits by closing the venous overflow line and the priming solution supplying line and then forcing the charged priming solution to be flowed; and a second circulating step for purging air from the priming solution by opening the priming solution supplying line and opening either one of the arterial overflow line and the venous overflow line.

Further, in a priming method for priming a blood purification apparatus, the priming solution charging step and the priming solution circulating step are performed by forcing the priming solution to be flowed from the bottom to the top of the blood purification means.

It is preferable that in a priming method for priming a blood purification apparatus, the priming solution charging step and the priming solution circulating step are performed by arranging the blood introducing port at the top of the blood purification means and the blood discharging port at the bottom of the blood purification means.

It is also preferable that in a priming method for priming a blood purification apparatus, a bubble detecting means for detecting bubbles in the blood circuits is arranged intermediately of the arterial blood circuit or the venous blood circuit, and the air purging in the second circulating step is performed with interlocking with the detection of bubbles by the bubble detecting means.

In a priming method for priming a blood purification apparatus, the priming method further includes a gas purging step for charging the dialysate route with dialysate by introducing the dialysate into the dialysate route of the blood purification means from the dialysate introducing line and discharging the dialysate from the dialysate discharging line, and wherein the gas purging step is automatically performed in the priming solution charging step and/or the priming solution circulating step.

According to the present invention, since the priming solution circulating step for forcing the charged priming solution to be flowed and circulated through the blood circuits by successively changing the driving speed of the blood pump after the priming solution charging step is performed, it is possible to firmly carry out the bubble purging during priming operation smoothly and in a short time.

According to the present invention, since the priming solution circulating step is performed by combining any two or more driving states of the blood pump of a normal rotation state, a reverse rotation state, and a stopped state, it is possible to set various flowing patterns of the priming solution in accordance with the priming conditions.

According to the present invention, since at least two different driving speeds of the blood pump can be set in the normal rotation state and the reverse rotation state, it is possible to more finely set the flowing pattern of the priming solution in accordance with the priming conditions.

According to the present invention, since the priming solution charging step is performed via such a first charging step as normally rotating the blood pump by a discharging quantity corresponding to a volume of the flow route in the venous blood circuit from the blood discharging port of the blood purification means (dialyzer) to the venous drip chamber, it is possible to commonly use this first charging step without changing the priming solution charging step although the blood purification apparatus of any one a wet type (in which the blood compartment and the dialysate compartment are previously filled with liquid such as purified-water) or a dry type (in which the blood compartment and the dialysate compartment are not filled with liquid) is connected to the blood circuits.

According to the present invention, since the priming method has the second circulating step for purging air from the priming solution by opening the priming solution supplying line and opening either one of the arterial overflow line and the venous overflow line, it is possible to more firmly perform air purging in the priming solution circulating step.

Based on the present invention, since the priming solution can be passed through the blood purification means (dialyzer) from its bottom to its top in the priming solution charging step and the priming solution circulating step, it is possible to firmly expel air bubbles mingled in the priming solution without inverting the blood purification means (dialyzer).

Based on the present invention, since the priming solution charging step and the priming solution circulating step are performed by arranging the blood introducing port at the top of the blood purification means (dialyzer) and the blood discharging port at the bottom of the blood purification means (dialyzer), it is possible to firmly expel air bubbles in the priming purification without inverting the blood purification means (dialyzer).

Based on the present invention, since the bubble detecting means for detecting bubbles in the blood circuits is arranged intermediately of the arterial blood circuit or the venous blood circuit, and the air purging in the second circulating step is performed with interlocking with the detection of bubbles by the bubble detecting means, it is possible to more firmly and smoothly perform air bubble purging in the priming solution circulating step.

According to the present invention, since the gas purging step is automatically performed in the priming solution charging step and/or the priming solution circulating step, it is possible to perform the priming solution charging step or the priming solution circulating step and the gas purging step in parallel with each other.

### BRIEF DESCRIPTION OF THE DRAWINGS

FIG. 1. A schematic view showing a dialysis apparatus (blood purification apparatus) of an embodiment of the present invention;

FIG. 2. A schematic view showing a condition in which a preparatory priming step is performed in the dialysis apparatus of FIG. 1;

FIG. 3. A schematic view showing a condition in which a first priming step is performed in the dialysis apparatus of FIG. 1;

FIG. 4. A schematic view showing a condition in which a second priming step is performed in the dialysis apparatus of FIG. 1;

FIG. 5. A schematic view showing a condition in which a third priming step is performed in the dialysis apparatus of FIG. 1;

FIG. 6. A schematic view showing a condition in which a priming solution circulating step is performed in the dialysis apparatus of FIG. 1;

FIG. 7. A schematic view showing a condition in which a first washing step is performed in the dialysis apparatus of FIG. 1;

FIG. 8. A schematic view showing a condition in which a second washing step is performed in the dialysis apparatus of FIG. 1;

FIG. 9. A schematic view showing a condition in which header bubble purging step is performed in the dialysis apparatus of FIG. 1;

FIG. 10. A time chart showing one example of a driving condition (driving pattern) of a blood pump in the circulating step in the dialysis apparatus of FIG. 1;

FIG. 11. A time chart showing one example of a driving condition (driving pattern) of a blood pump in the circulating step in the dialysis apparatus of FIG. 1;

FIG. 12. A time chart showing one example of a driving condition (driving pattern) of a blood pump in the circulating step in the dialysis apparatus of FIG. 1;

FIG. 13. A time chart showing one example of a driving condition (driving pattern) of a blood pump in the circulating step in the dialysis apparatus of FIG. 1;

FIG. 14. A schematic view showing a dialysis apparatus of another embodiment of the present invention;

FIG. 15. A schematic view showing a dialysis apparatus of another embodiment of the present invention;

FIG. 16. A schematic view showing a dialysis apparatus of another embodiment of the present invention;

FIG. 17. A schematic view showing a condition in which a first charging step is performed in a dialysis apparatus (blood purification apparatus) of another embodiment of the present invention;

FIG. 18. A schematic view showing a condition in which a second charging step is performed in the dialysis apparatus of FIG. 17;

FIG. 19. A schematic view showing a condition in which a third charging step is performed in the dialysis apparatus of FIG. 17;

FIG. 20. A schematic view showing a condition in which a fourth charging step is performed in the dialysis apparatus of FIG. 17;

FIG. 21. A schematic view showing a condition in which a gas purge step is performed in the dialysis apparatus of FIG. 17;

FIG. 22. A schematic view showing a condition in which a first circulating step is performed in the dialysis apparatus of FIG. 17;

FIG. 23. A schematic view showing a condition in which an other first circulating step is performed in the dialysis apparatus of FIG. 17;

FIG. 24. A schematic view showing a condition in which a further first circulating step is performed in the dialysis apparatus of FIG. 17;

FIG. 25. A schematic view showing a condition in which a still further first circulating step is performed in the dialysis apparatus of FIG. 17;

FIG. 26. A schematic view showing a condition in which the third charging step and the fourth charging step are simultaneously performed in the dialysis apparatus of FIG. 17;

FIG. 27. A schematic view showing a condition in which the first charging step and the gas purge step are simultaneously performed in the dialysis apparatus of FIG. 17; and

FIG. 28. A graph showing a comparison of a time required for priming of the present invention and a comparative example.

### DETAILED DESCRIPTION OF THE INVENTION

Preferable embodiments of the present invention will be hereinafter described with reference to the drawings.

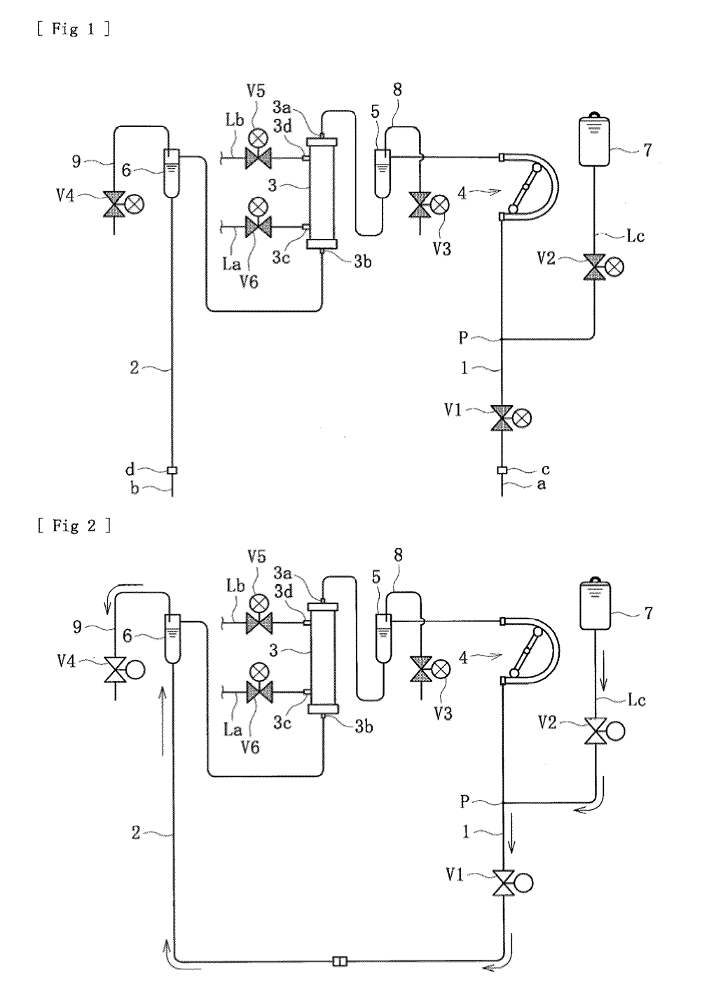

A blood purification apparatus of an embodiment of the present invention is a dialyzer for performing the dialysis and mainly comprising, as shown in FIG. 1, blood circuits including an arterial blood circuit 1 and a venous blood circuit 2, a dialyzer (blood purification means) 3 for purifying blood flowing the blood circuits interposed between the arterial blood circuit 1 and the venous blood circuit 2, a blood pump 4 of squeezing-tube type arranged in the arterial blood circuit 1, an arterial drip chamber 5 and a venous drip chamber 6 arranged respectively in the arterial blood circuit 1 and the venous blood circuit 2, a containing means 7 for containing saline as priming solution, and a priming solution supplying line Lc for connecting the containing means 7 and the arterial blood circuit 1.

An arterial needle “a” is adapted to be connected to a tip of the arterial blood circuit 1 via a connector “c” and the blood pump 4 and the arterial drip chamber 5 are arranged intermediately of the arterial blood circuit 1. On the other hand, in the venous blood circuit 2, a venous needle “b” is adapted to be connected to a tip of the venous blood circuit 2 via a connector “d” and the venous drip chamber 6 is arranged intermediately of the venous blood circuit 2. After having punctured the arterial needle “a” and the venous needle “b” respectively into the shunt (Vascular access) of a patient, when starting the blood pump 4, blood of the patient flows through the arterial blood circuit 1 to the dialyzer 3 and is purified by the dialyzer 3, and then after having removed air bubbles from the blood in the venous drip chamber 6, the blood is returned to the venous of a patient through the venous blood circuit 2. That is, blood of a patient can be purified by the dialyzer 3 with being extracorporeally circulated through from the tip of the arterial blood circuit 1 to the tip of the venous blood circuit 2.

An arterial overflow line 8 and a venous overflow line 9 extend respectively from the tops (air layer sides) of the arterial drip chamber 5 and the venous drip chamber 6 and electromagnetic valves V3 and V4 are arranged respectively on the overflow lines 8 and 9. That is, the arterial overflow line 8 and the venous overflow line 9 extend from the tops of the arterial drip chamber 5 and the venous drip chamber 6 so as to open the air layer sides of them to the atmosphere and are adapted to be opened and closed by the electromagnetic valves V3 and V4.

A casing of the dialyzer 3 is provided with a blood introducing port 3a, a blood discharging port 3b, a dialysate introducing port 3c, and a dialysate discharging port 3d and the arterial blood circuit 1 is connected to the blood introducing port 3a and the venous blood circuit 2 is connected to the blood discharging port 3b. In addition a dialysate introducing line La extending from a dialysate supplying device (not shown) is connected to the dialysate introducing port 3c and a dialysate discharging line Lb also extending from the body of the dialysis apparatus is connected to the dialysate discharging port 3d.

A large number of hollow fibers are contained in the dialyzer 3 and these hollow fibers form a blood purification membrane. Formed within the dialyzer 3 is a blood route through which blood of a patient flows and a dialysate route through which the dialysate flows. Each hollow fiber is formed with micro pores and forms a hollow fiber membrane and waste products in blood are passed through the follow fiber membrane into the dialysate.

A duplex pump (not shown) is arranged in the body of the dialysis apparatus with bridging the dialysate introducing line La and the dialysate discharging line Lb and a ultrafiltration pump (not shown) is also arranged in the body of the dialysis apparatus for removing water from blood of a patient flowing in the dialyzer 3. As described above, one end of the dialysate introducing line La is connected to the dialysate introducing port 3c and its other end is connected to the dialysate supplying device for preparing dialysate of predetermined concentration. Also, one end of the dialysate discharging line Lb is connected to the dialysate discharging port 3d of the dialyzer 3 and its other end is connected to a discharging device (not shown). Accordingly, the dialysate supplied from the dialysate supplying device to the dialyzer 3 through the dialysate introducing line La is sent to the discharging device through the dialysate discharging line Lb from the dialysate discharging port 3d of the dialyzer 3.

An electromagnetic valve V6 is arranged intermediately (between the duplex pump and the dialyzer 3) of the dialysate introducing line La for opening and closing the line La, an electromagnetic valve V5 is arranged intermediately (between the duplex pump and the dialyzer 3) of the dialysate discharging line Lb for opening and closing the line Lb, and an electromagnetic valve V1 is arranged intermediately of the arterial blood circuit 1 for opening and closing the blood circuit 1.

The opening and closing operations of these electromagnetic valves V1, V5 and V6 (similarly to the electromagnetic valves V2 through V4) can be controlled by a control means such as a microcomputer. That is, the microcomputer is connected not only to the electromagnetic valves V1, V5 and V6 but also the V2 arranged intermediately of the priming solution supplying line Lc, the electromagnetic valve V3 arranged intermediately of the arterial overflow line 8 extending from the top (air layer side) of the arterial drip chamber 5 and the electromagnetic valve V4 arranged intermediately of the venous overflow line 9 extending from the top (air layer side) of the venous drip chamber 6 and, can control the opening and closing operations of these electromagnetic valves.

The physiological saline solution containing means 7 (a so-called saline bag) comprises a flexible transparent container for containing a predetermined volume of the saline (i.e. priming solution) and is adapted to be hung on the tip of a standing pole (not shown) mounted on the dialysis apparatus. The priming solution supply line Lc is connected to a junction (connecting portion) P between the arterial needle “a” at the tip of the arterial blood circuit 1 and the blood pump 4 for supplying the priming solution in the containing means 7 to the blood circuits.

The dialysis apparatus (blood purification apparatus) of the present invention can connect the tips (respectively the connectors “c” and “d”) of the arterial blood circuit 1 and the venous blood circuit 2 to form a closed communicating circuit for circulating the priming solution (saline) to wash the flowing lines of the blood circuits 1 and 2 during the priming step. As previously described, the arterial overflow line 8 and the venous overflow line 9 are arranged to overflow an excessive priming solution supplied from the priming solution supplying line Lc.

Then the priming step is performed an embodiment of the dialysis apparatus of the present invention. As shown in FIG. 2, the connectors “c” and “d” are connected, with arranging the blood introducing port 3a of the dialyzer 3 to be directed to top side, to form the continuously closed circuit for circulating the priming solution and then the electromagnetic valves V2, V1 and V4 are set at the opened position and the other electromagnetic valves V3, V5 and V6 are set at the closed position.

As shown in FIG. 2 the priming solution in the containing means 7 flows down by gravity caused by the head drop to the venous drip chamber 6 and is discharged therefrom through the venous overflow line 9 extending from the top of the venous drip chamber 6. This enables to assure the solution level in the venous drip chamber 6 and this step is referred to as a “preparatory priming step”.

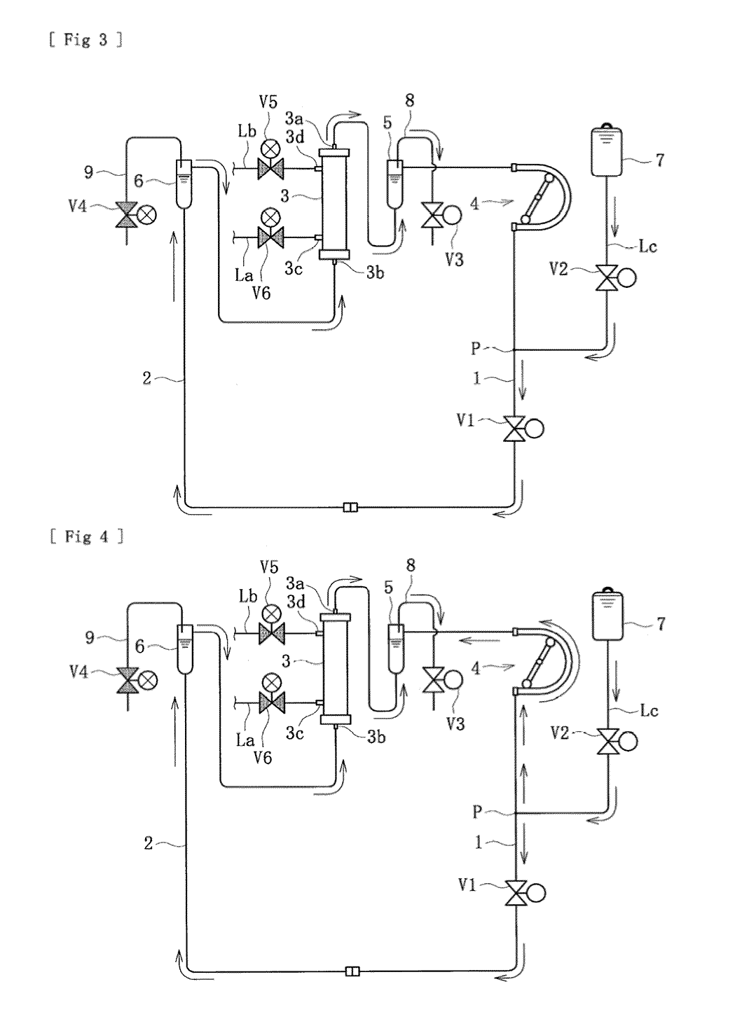

Then the apparatus is shifted to a condition shown in FIG. 3 in which the electromagnetic valve V4 is closed and the electromagnetic valve V3 is opened. The priming solution in the containing means 7 flows down by gravity caused by the head drop to the arterial drip chamber 5 and is discharged therefrom through the arterial overflow line 8 extending from the top of the arterial drip chamber 5. During this step, the priming solution flows to the venous blood circuit 2 by gravity and is circulated in the blood circuits 1 and 2 and finally discharged from the overflow line 8 through the blood discharging port 3b and then the blood introducing port 3a. This step is referred to as a “first priming step”.

Then the blood pump 4 is actuated and the priming solution is forced to be flowed also in the arterial blood circuit 1 between the junction P and the arterial drip chamber 5 and discharged therefrom through the arterial overflow line 8 as shown in FIG. 4. This step is referred to as a “second priming step”. During this step, the blood pump 4 is controlled so that the priming solution can flow from the junction P to both sides, i.e. to the blood pump 4 and the venous drip chamber 6.

The first priming step and the second priming step form a “priming solution charging step” of the present invention (the preparatory priming step may be included in this priming solution charging step) in which the priming solution is fed into the blood circuits 1 and 2 to charge them with the tips of the blood circuits 1 and 2 being connected to be communicated each other. In this priming solution charging step, it is sufficient that the priming solution charges the blood circuits 1 and 2 and any other step may be used than the steps including the first and second priming steps.

Since the priming solution is discharged from the arterial overflow line 8 in the first and second priming steps, it is possible assure the solution level in the arterial drip chamber 5. In addition since the priming solution flows through the first and second priming steps, it is possible to fill the blood flow route of the artery-side blood circuit 1, the venous blood circuit 2 and the dialyzer 3 with the priming solution.

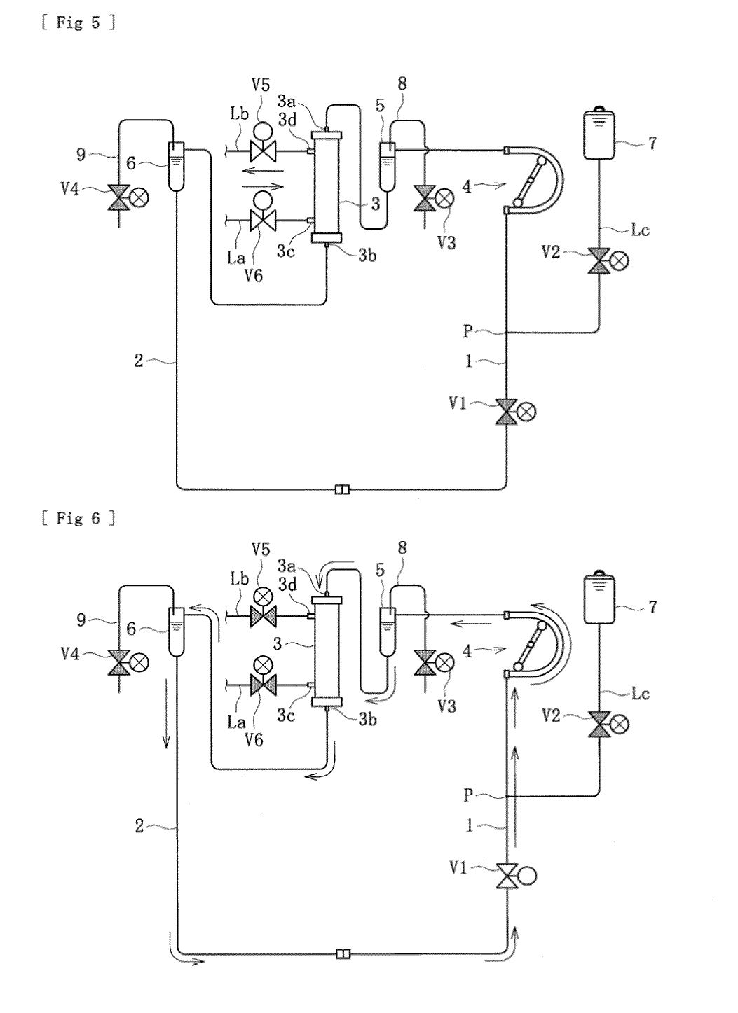

Then the apparatus is shifted to a next step in which the blood pump 4 is stopped, the electromagnetic valves V1 through V4 are closed and the electromagnetic valves V5 and V6 are opened as shown in FIG. 5. During which the duplex pump is actuated to introduce the dialysate into the dialysate flow route within the dialyzer 3 from the dialysate introducing line La and discharge it from the dialysate flow route and thus the dialysate flow route is charged with the dialysate. This step is referred to as a “third priming step”. After this third priming step, the apparatus is in a condition in which the dialysate flow route has been filled with the dialysate.

After the third priming step, the priming solution i.e. saline is circulated through the closed circuit by closing the electromagnetic valves V5 and V6 (the closed condition of the electromagnetic valves V2, V3 and V4 is still maintained), opening the electromagnetic valve V1 and actuating the blood pump 4 as shown in FIG. 6. The circulation of the charged priming solution causes circulation of air bubbles remaining in the blood flow route and enables the bubbles to be trapped by the arterial drip chamber 5 and the venous drip chamber 6. This step is referred to as a “circulating step”.

According to the circulating step of the present invention, the charged priming solution is circulated in the blood circuits 1 and 2 with its flowing speed being changed by successively changing the driving speed of the blood pump 4 after the priming solution charging step (“step for supplying and charging the blood circuits with the priming solution” including the preparatory priming step and the first and second priming steps).

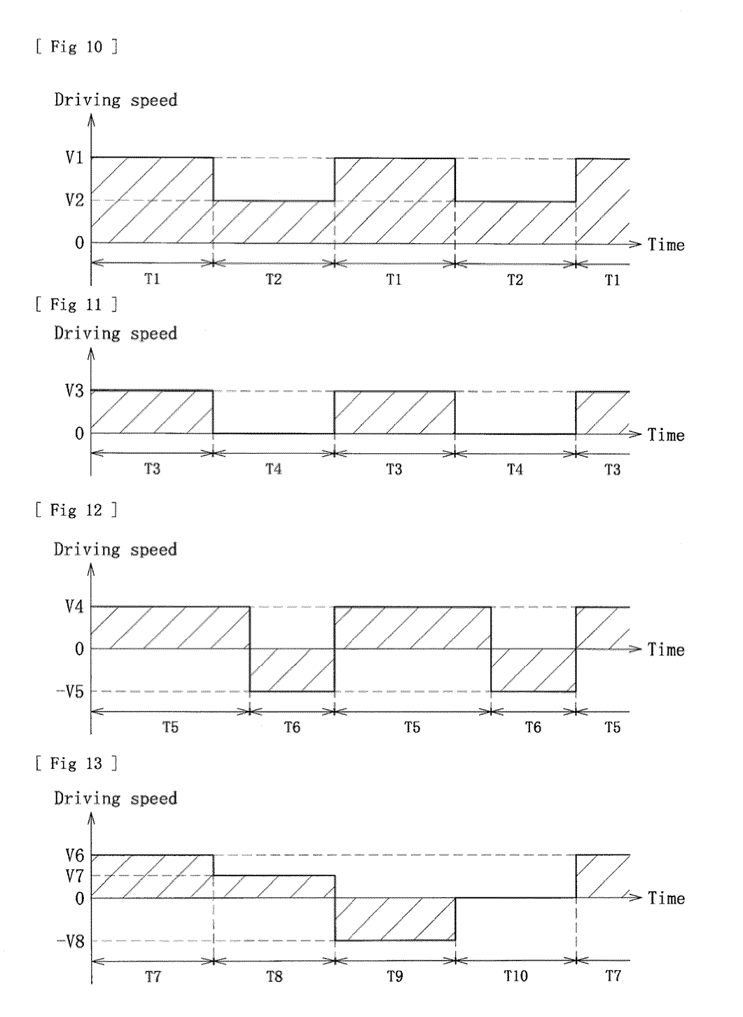

More in particularly, the priming solution circulating step is performed by combining any two or more driving states of the blood pump 4 of a normal rotation state (state of positive driving speed of the pump 4), a reverse rotation state (state of negative driving speed of the pump 4), and a stopped state (state of zero driving speed of the pump 4). In addition, in the normal rotation state and the reverse rotation state, it is possible to set at least two different driving speed of the pump 4. The priming solution circulating step of the present invention will be hereinafter described with reference to FIG. 10 through FIG. 13.

In an example of the priming solution circulating step shown in FIG. 10, when the driving speed of the blood pump 4 is set so that it is alternately repeated as it has a time T1 at a driving speed V1 (normal rotation) and a time T2 at a driving speed V2 (normal rotation), the priming solution charged in the priming solution charging step flows in one direction with varying its flowing speed. Accordingly the air bubbles adhered on the inner wall surface of the blood circuits can be easily broken away therefrom and entrained by the priming solution as compared with an occasion in which the priming solution flows at a constant speed and the entrained bubbles are trapped by the arterial drip chamber 5 or the venous drip chamber 6. In this circulating step, the times T1 and T2 may be same or different each other.

In an other example of the priming solution circulating step shown in FIG. 11, when the driving speed of the blood pump 4 is set so that it is alternately repeated as it has a time T3 at a driving speed V3 (normal rotation) and a time T4 at a zero driving speed, the priming solution charged in the priming solution charging step flows intermittently in one direction. Accordingly the air bubbles adhered on the inner wall surface can be easily broken away therefrom and entrained by the priming solution as compared with an occasion in which the priming liquid flows at a constant speed and the entrained bubbles are trapped by the arterial drip chamber 5 or the venous drip chamber 6. In this circulating step, the times T3 and T4 may be same or different each other.

In an other example of the priming venous circulating step shown in FIG. 12, when the driving speed of the blood pump 4 is set so that it is alternately repeated as it has a time T5 at a driving speed V4 (normal rotation) and a time T6 at a driving speed −V5 (reverse rotation), the priming solution charged in the priming solution charging step repeatedly flows in one direction and in a reverse direction and flows forward as a whole in the circulating step. Since in this case the priming solution flows not only in one direction but also in the reverse direction, the air bubbles adhered on the inner wall surface can be more easily broken away therefrom and entrained by the priming and the entrained bubbles are trapped by the arterial drip chamber 5 or the venous drip chamber 6. In this circulating step, the times T5 and T6 may be same or different each other.

In an other example of the priming solution circulating step shown in FIG. 13, when the driving speed of the blood pump 4 is set so that it is alternately repeated as it has a time T7 at a driving speed V6 (normal rotation), a time T8 at a driving speed V7 (normal rotation), a time T9 at a driving speed −V8 (reverse rotation), and a time T10 at a zero driving speed, the priming solution charged in the priming solution charging step repeatedly flows in one direction at a high speed (V6) and a low speed (V7) and then flows in a reverse direction (−V8) and then is once stopped, and the priming solution flows forward as a whole in the circulating step. Since in this case the flowing speed is successively changed and the priming solution flows not only in one direction but also in the reverse direction, the air bubbles adhered on the inner wall surface can be more easily broken away therefrom and entrained by the priming and the entrained bubbles are trapped by the arterial drip chamber 5 or the venous drip chamber 6. In this circulating step, the times T7 through T10 may be same or different each other.

As described above, it is sufficient that the priming solution circulating step is performed by combining any two or more driving states of the blood pump 4 of a normal rotation state, a reverse rotation state, and a stopped state and the combination can be appropriately determined in consideration e.g. of priming conditions. In addition, it is also sufficient that at least two driving speed of the pump 4 can be set in the normal rotation condition and the reverse rotation condition and it is possible to set more than two driving speeds.

The driving speeds V1 through V8 in FIG. 10 through FIG. 13 may be set within a discharging capacity of the blood pump 4 (in general 0˜±600 mL/min). However it is preferable to set the driving speed of the pump 4 at 0˜±400 mL/min in order to reduce entrapping of air into the priming solution at the arterial and venous drip chambers 5 and 6. The times T1 through T10 may be set so that an amount of saline (priming solution) defined in the instructions manual (accompanying document) for a dialyzer to be used can be obtained within a desired time and also the air purging effect can be obtained in consideration of the combination of the T1 through T10 and the driving speeds V1 through V8 and the opening duration of the arterial and venous overflow lines 8 and 9. These settings will be easily considered by those skilled in the art without disclosing concrete values in this specification.

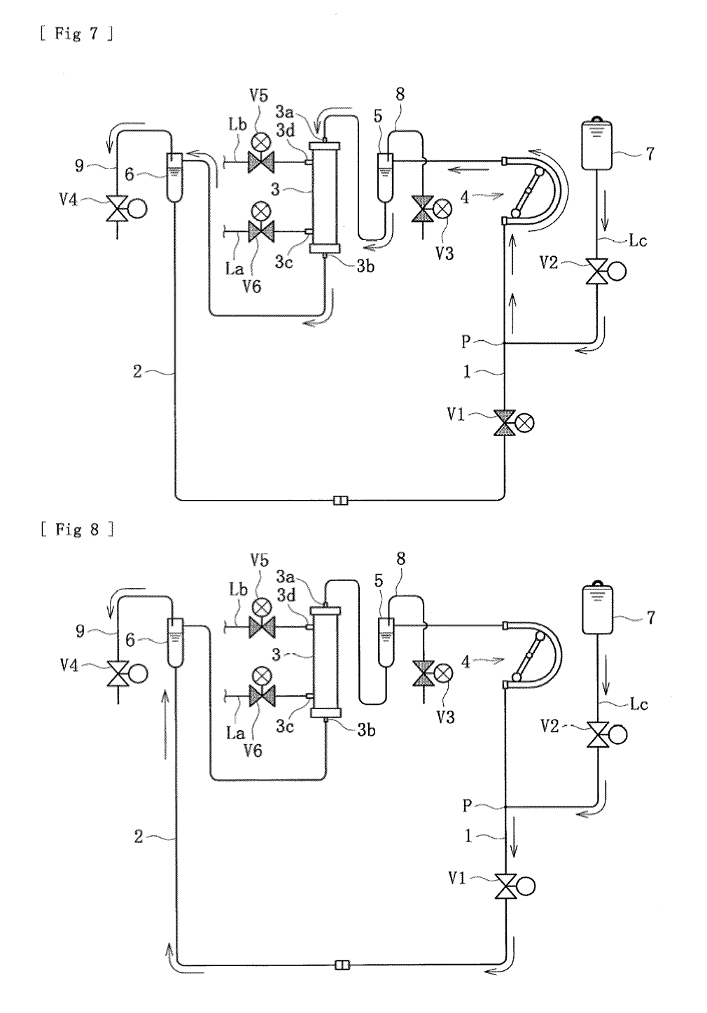

Then the dialysis apparatus is shifted to a condition shown in FIG. 7 in which the electromagnetic valves V2 and V4 are opened to supply again saline (priming solution) in the containing means 7 into the blood circuits 1 and 2 and to discharge an excessive priming solution from the overflow line 9 extending from the venous drip chamber 6. Since the electromagnetic valve V1 is closed, it is possible to surely wash by the fresh priming solution in the containing means 7 the flowing route from the junction P to the venous drip chamber 6 through the arterial drip chamber 5 and the dialyzer 3. This step is referred to as a “first washing step”.

Then the dialysis apparatus is shifted to a condition shown in FIG. 8 in which the blood pump 4 is stopped and the electromagnetic valve V1 is opened; the priming solution in the containing means 7 flows down by gravity caused by the head drop to the venous drip chamber 6 and is discharged therefrom through the venous overflow line 9 extending from the top of the venous drip chamber 6. This reliably enables washing by the fresh priming solution in the containing means 7 in the flowing route from the junction P to the venous drip chamber 6 without passing through the dialyzer 3. This step is referred to as a “second washing step”.

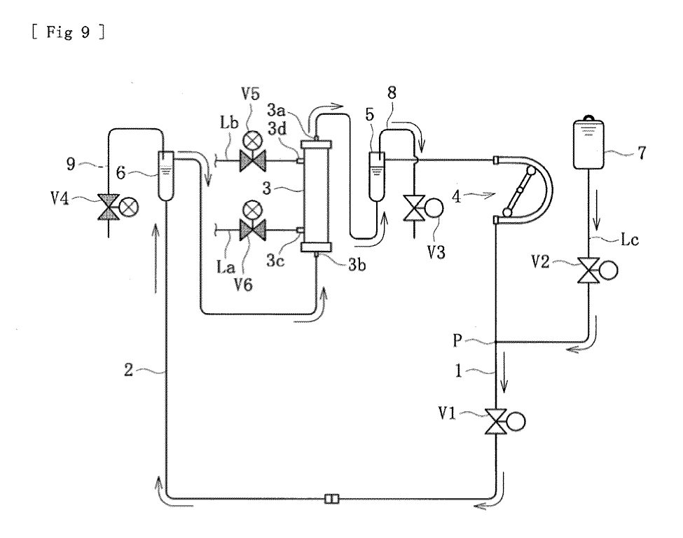

Finally the dialysis apparatus is shifted to a condition shown in FIG. 9 in which the electromagnetic valve V4 is closed and the electromagnetic valve V3 is opened to flow down the priming solution in the containing means 7 by gravity caused by the head drop to the arterial drip chamber 5 and to discharged therefrom through the arterial overflow line 8 extending from the top of the arterial drip chamber 5. This enables to discharge air bubbles remained near the blood introducing port 3a of the dialyzer 3 from the arterial overflow line 8. This step is referred to as a “header bubble purging step”.

As described above, according to the present invention since the priming solution circulating step is performed by forcing the priming solution charged by successively changing the driving speed of the blood pump 4 after the priming solution charging step (including the normal rotation condition, reverse rotation condition and stopped condition of the blood pump) to be flowed and circulated in the blood circuits 1 and 2, it is possible to perform the bubble purging in the priming operation more smoothly and in a shorter time as compared with the conventional manner in which the priming solution is flowed at a constant speed.

In addition, since the priming solution circulating is performed by combining any two or more driving states of the blood pump 4 of a normal rotation state, a reverse rotation state, and a stopped state, it is possible to set various flowing patterns of the priming solution in accordance with the priming conditions. Furthermore, since at least two different driving speed can be set in the normal rotation condition and the reverse rotation condition, it is possible to more finely set the flowing patterns of the priming solution in accordance with the priming conditions.

It is also possible to perform the washing and priming of portions through which blood and dialysate flow route during dialysis and thus to surely discharge bubbles to outside by a series of steps such as the preparatory priming step, the first priming step, the second priming step, the third priming step, the priming liquid circulating step, the first washing step, the second washing step, and the header bubble purging step. In addition, since all the priming steps are performed with the blood introducing port 3a of the dialyzer 3 being directed upward without requiring any upset operation of the dialyzer 3, it is possible to easily automatize the priming step and to achieve a quick and sure bubble purging in the dialyzer 3.

In addition, the charged priming solution is circulated by actuating the blood pump 4 after the first and second priming steps, it is possible to more surely remove the bubbles from the priming solution. Furthermore, since the priming solution can be discharged through the arterial overflow line 8 and venous overflow line 9, it is possible to apply the present invention to a dialysis apparatus of the prior art and to obtain a dialysis apparatus of the present invention by additionally providing the dialysis apparatus of the prior art with the overflow lines.

It is preferable to use a drip chamber at least of the arterial not having inside any mesh in order to discharge the bubbles outside more smoothly. In addition, when the arterial drip chamber 5 and the venous drip chamber 6 are provided with pressure monitoring lines for detecting the pressure in the air layer sides of the drip chambers 5 and 6, it is preferable to arrange the base ends of the pressure monitoring lines at positions higher than those of the overflow lines 8 and 9 in order to prevent flowing of the priming solution into the pressure monitoring lines during overflowing of the priming solution.

Now a further embodiment of the blood purification apparatus (dialyzer) of the present invention will be described with reference to FIGS. 17 through 23. The same reference numerals are used herein for designating the same parts as those having same functions used in the above embodiment and accordingly detailed descriptions of the parts will be omitted.

Similarly to the above embodiment, the blood purification apparatus of this embodiment mainly comprises blood circuits including the arterial blood circuit 1 and the venous blood circuit 2, the dialyzer (blood purification means) 3, peristaltic blood pump 4, the arterial drip chamber 5, the venous drip chamber 6, the priming solution containing means 7, and priming solution supplying line Lc.

In this embodiment, an electromagnetic valve V1′ is arranged on the venous blood circuit 2 in place of or together with the electromagnetic valve V1. In addition, a bubble detecting means 10 for detecting bubbles in the venous blood circuit 2 is arranged on the venous blood circuit 2 downstream of the venous drip chamber 6 (between the venous drip chamber 6 and the electromagnetic valve V1). The bubble detecting means may be arranged in any position of the arterial blood circuit 1.

The bubble detecting means 10 is a so-called bubble sensor for detecting bubbles in the arterial blood circuit 1 or the venous blood circuit and has for example a structure for detecting the bubbles by irradiate the ultra sonic wave onto tubes forming the arterial blood circuit 1 or the venous blood circuit 2 and detecting its damping or absorption factor. An other structure may be used for the bubble detecting means 10.

Then the priming step performed in the dialysis apparatus of an embodiment will be described.

As shown in FIG. 17, the priming is performed with keeping the blood introducing port 3a of the dialyzer 3 upward by holding the dialyzer 3 using a dialyzer holder (not shown), connecting the connectors “c” and “d” to communicate the blood circuits 1 and 2, then opening the electromagnetic valve V1′, and closing other electromagnetic valves V2 through V6.

Then the blood pump 4 is normally rotated by a discharging volume corresponding to a volume of the flowing route of the venous blood circuit 2 from the blood discharging port 3b of the dialyzer 3 to the venous drip chamber 6. This step is for convenience referred to as “first charging step”. It should be noted that the “normal rotation” of the blood pump 4 is defined as a rotation of the pump 4 to a direction in which blood of a patient is sent from the arterial blood circuit 1 to the tip of the venous blood circuit 2 via the dialyzer 3 during performing the extracorporeal circulation of the blood of a patient in dialysis.

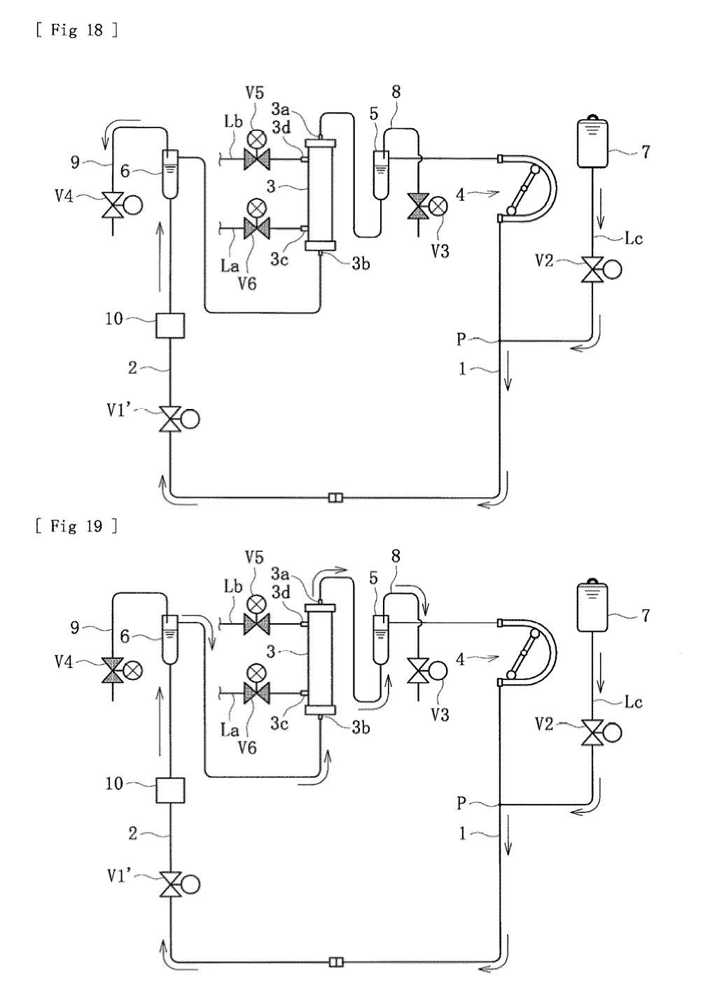

Then as shown in FIG. 18, the blood pump 4 is stopped, the venous overflow line 9 is opened (closed condition of the arterial overflow line 8 and opened condition of the electromagnetic valve V1′ are maintained), and the electromagnetic valve V2 is opened to supply the priming solution from the priming solution supplying line Lc. Thus the priming solution can flow from the junction P of the arterial blood circuit 1 to the venous drip chamber 6 of the venous blood circuit 2 via the connection of the connectors “c” and “d” of the tips of the arterial and venous blood circuits 1 and 2 and is finally discharged from the venous overflow line 9. This step is referred to as a “second charging step”.

That is, in the second charging step the priming solution (saline) flows to the venous drip chamber 6 by gravity of the priming solution caused by the head drop and finally discharged through the venous overflow line 9 extending from the top of the venous drip chamber 6 with stopping the blood pump 4 and opening the electromagnetic valve V2. Thus it is possible to assure the solution level in the venous drip chamber 6.

Then as shown in FIG. 19, with keeping the blood pump 4 in the stopped condition, the electromagnetic valve V4 is closed and the electromagnetic valve V3 is opened and the venous overflow line 9 is closed and the arterial overflow line 8 is opened and the electromagnetic valve V2 is opened to supply the priming solution from the priming solution supplying line Lc. Thus the priming solution can flow from the junction P of the arterial blood circuit 1 to the arterial drip chamber 5 of the arterial blood circuit 1 via the connection of the connectors “c” and “d” of the tips of the arterial and venous blood circuits 1 and 2 and is finally discharged from the arterial overflow line 8. This step is referred to as a “third charging step”.

That is, similarly to the second charging step, also in the third charging step, the priming solution (saline) flows to the arterial drip chamber 5 by gravity of the priming solution caused by the head drop and finally discharged through the arterial overflow line 8 extending from the top of the arterial drip chamber 5 with closing the electromagnetic valve V4 and opening the electromagnetic valve V3. As clearly understood, in the third charging step, the priming solution is supplied from the priming solution supplying line Lc and flowed into the venous blood circuit 2 by gravity and charged in the flowing route with being discharged from the arterial overflow line 8 through the blood discharging port 3b and then the blood introducing port 3a of the dialyzer 3.

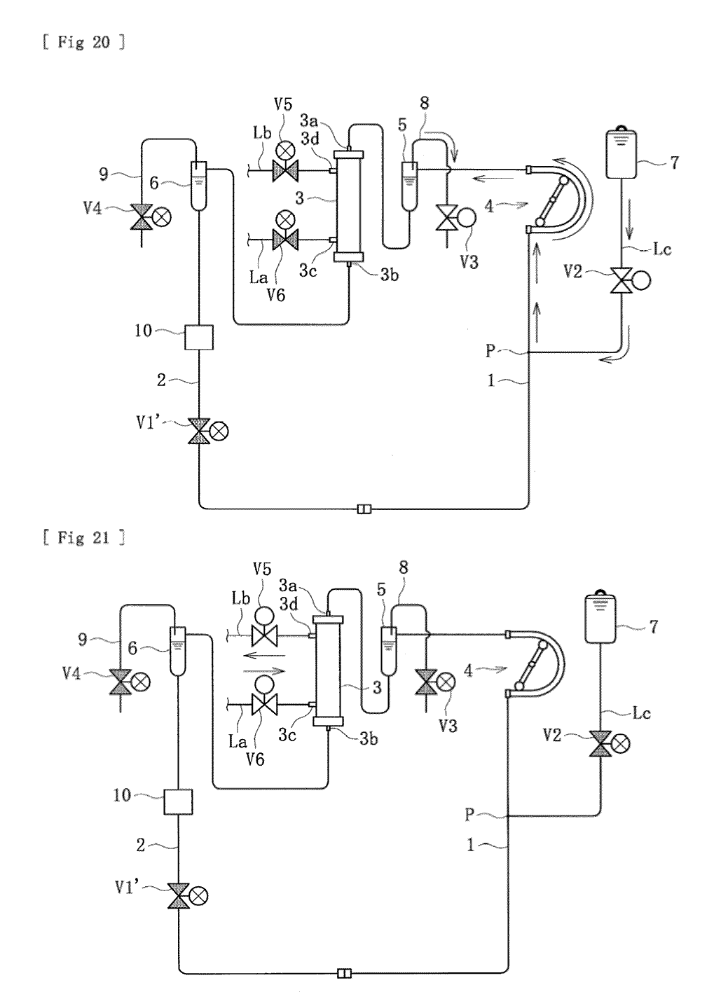

As shown in FIG. 20, the priming solution is then flowed from the junction P of the arterial blood circuit 1 to the arterial drip chamber 5 of the arterial blood circuit 1 via the blood pump 4 and finally discharged from the arterial overflow line 8 with closing the electromagnetic valve V1′ and actuating again the blood pump 4 in a normal rotation direction. This step is referred to as a “fourth charging step”. It should be noted that the first through fourth charging steps form the “priming solution charging step” of the present invention.

That is, in the fourth charging step, the priming solution (saline) is flowed in a direction reverse to those in the second and third charging steps and discharged from the arterial overflow line 8 and thus charge its flowing route. In this case, it is possible to control the driving speed of the blood pump 4 so that the priming solution can flow from the junction P to both sides, i.e. to the blood pump 4 and the venous drip chamber 6 with keeping the opening condition of the electromagnetic valve V1′.

Furthermore the blood purification apparatus is shifted to a condition in which the electromagnetic valves V1′, V2, V3 and V4 are closed, the blood pump 4 is stopped and the electromagnetic valves V5 and V6 are opened as shown in FIG. 21. Under the circumstances, the duplex pump is driven to introduce the dialysate into the dialysate route from the dialysate introducing line La and to charge the dialysate route with the dialysate. This step is referred to as a “gas purge step”. The dialysate route is filled with the dialysate after this gas purge step.

The gas purge step may be performed after the priming solution charging step (first through fourth charging steps) have been completed or after the priming solution circulating steps have been completed. Furthermore, the gas purge step may be automatically performed intermediately of the priming solution charging step or the priming solution circulating step at any timing or simultaneously with these steps. In this case the gas purge step may be performed in parallel with the priming solution charging step or circulating step.

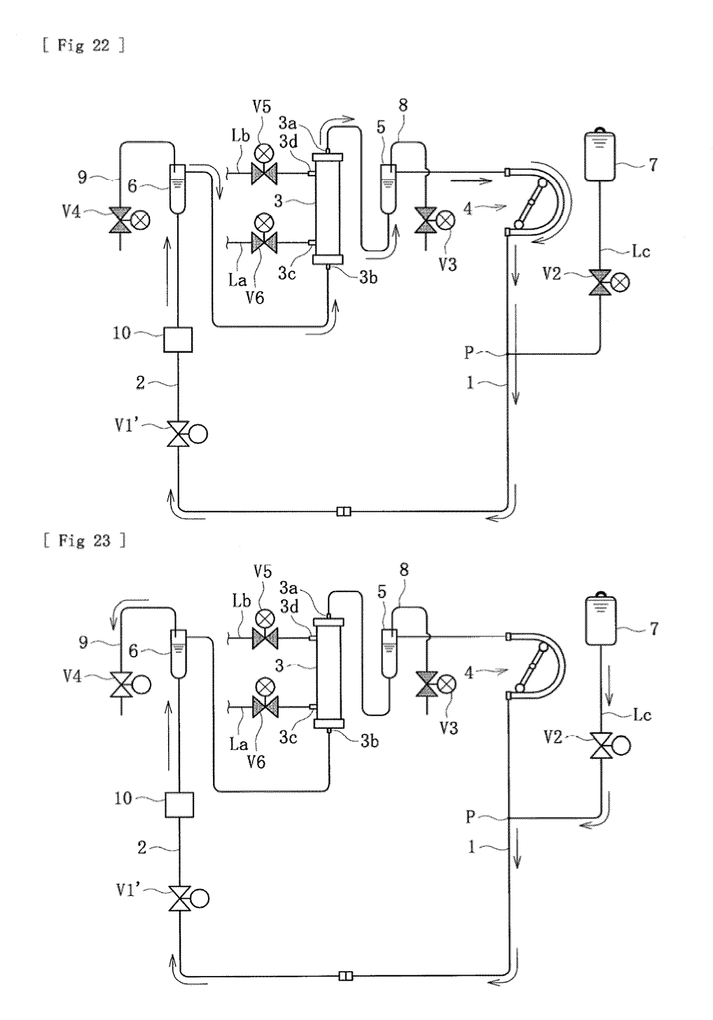

Then the blood purification apparatus is shifted to a condition in which the electromagnetic valves V1′ is opened, the electromagnetic valves V2 through V6 are closed, and the blood pump 4 is driven in a reverse direction as shown in FIG. 22. Accordingly the arterial overflow line 8, the venous overflow line 9 and the priming solution supplying line Lc are closed and thus the charged priming solution can be circulated in the blood circuits (arterial blood circuit 1 and the venous blood circuit 2). This step is referred to as a “first circulating step”. In this first circulating step, it is possible to quickly discharge air remained within the blood circuits 1 and 2 and the dialyzer 3 to the arterial or venous drip chambers 5 or 6 by driving the blood pump 4 with successively changing its driving speed. The air sent to the arterial or venous drip chambers 5 or 6 can be trapped therein. As previously described the gas purge step may be performed simultaneously with the first circulating step as shown in FIG. 27.

After the first circulating step, the blood purification apparatus is shifted to a condition shown in FIG. 23 in which the blood pump 4 is stopped and the electromagnetic valves V1′, V2 and V4 are opened. Since the priming solution supplying line Lc is opened and the venous overflow line 9 is also opened, the priming solution can be discharged together with included air through the venous overflow line 9 (air purging). This air purging step is referred to as a “second circulating step”.

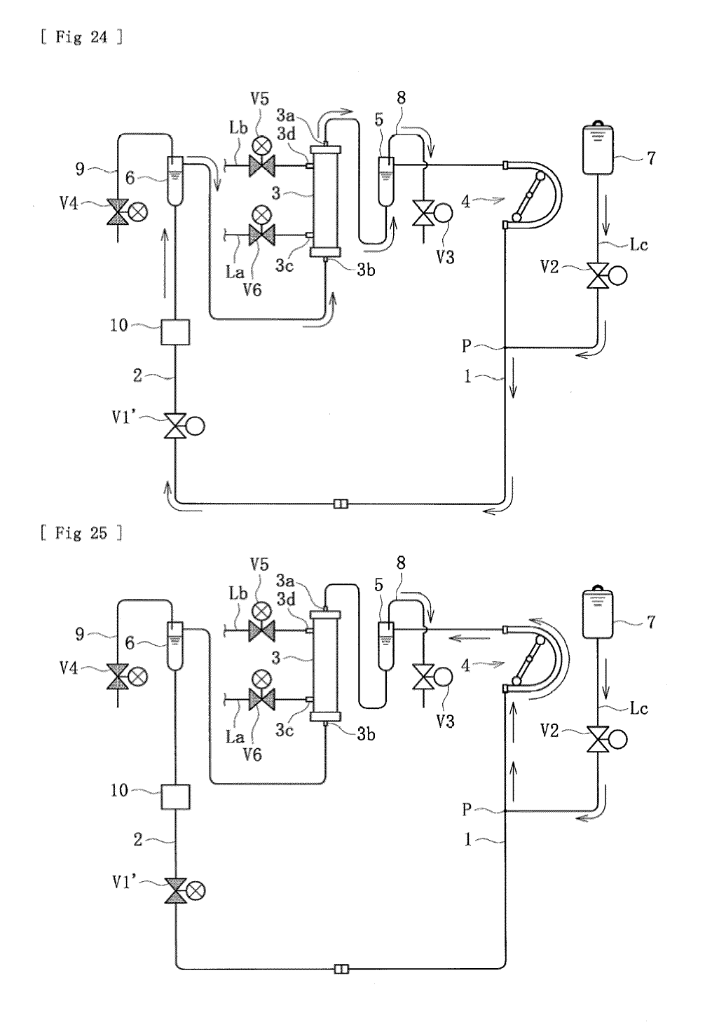

In the second circulating step, it is possible to quickly discharge air remained within the blood circuits 1 and 2 and the dialyzer 3 to the arterial or venous drip chambers 5 or 6 by driving the blood pump 4 with successively changing its driving speed. The air sent to the arterial or venous drip chambers 5 or 6 can be trapped therein. The second circulating step may be performed in other arrangements of the blood purification apparatus. For example the air purging may be performed through the arterial overflow line 8 in an arrangement shown in FIG. 24 in which the venous overflow line 9 is closed and the arterial overflow line 8 is opened or the air purging may be performed through the arterial overflow line 8 in an arrangement shown in FIG. 25 in which the blood pump 4 is driven in a normal direction and the arterial overflow line 8 is opened (the electromagnetic valve V1′ may be closed as shown in FIG. 25 or opened).

The first and second circulating steps form the “circulating step” of the present invention. According to this embodiment of the present invention, since it has the second circulating step for performing the air purging in which the priming solution supplying line Lc is opened, and either one of the arterial or venous overflow line 8 or 9 is opened, it is possible to more surely perform the air purging in the circulating step.

In addition since the bubble detecting means 10 is arranged intermediately of the venous blood circuit 2 (it may be arranged on the arterial blood circuit 1), it is preferable that the air purging in the second circulating step is performed interlocking with the detection of bubbles by the bubble detecting means 10. This makes it possible to perform the air purging in the circulating step more firmly and smoothly.

According to the present invention, since the priming solution charging step includes the first charging step in which the blood pump 4 is normally rotated by a discharging volume corresponding to a volume of the flowing route of the venous blood circuit 2 from the blood discharging port 3b of the dialyzer 3 to the venous drip chamber 6, it is possible to fill the blood route from the blood discharging port 3b to the venous drip chamber 6 with the charging solution within the dialyzer 3 in a case of a so-called wet type (type in which the blood compartment and the dialysate compartment are previously charged with the purified water) (air in the blood circuits will enter into the dialyzer through the blood introducing port 3a).

Since the blood route from the priming solution supplying line Lc to the blood discharging port 3b is filled with the priming solution and dialyzer charging solution when the venous drip chamber 6 is filled with the priming solution in next second charging step, it is possible to minimize an amount of air originally existed in the blood circuits to be sent to the dialyzer 3 and thus to reduce a time in the circulating step after the air purging.

In a case of dry type (type in which the blood compartment and the dialysate compartment are not filled with purified water), since the dialyzer 3 is originally filled with air, no matter what happened in the charging step if the first charging step is performed. Accordingly it is possible to perform the priming solution charging step in a common way without adding any change to the priming solution charging step although any form of dialyzer is connected to the blood circuits.

According to the present invention, since the priming solution flows from the bottom to the top of the dialyzer 3 in the first through fourth charging steps (priming solution charging step) and circulating step (first and second circulating steps), it is possible to fill the dialyzer 3 with the priming solution and to surely purge air included in the priming solution without inverting the dialyzer 3. In addition according to the present invention, since it is unnecessary to move (invert) the drip chambers 5 and 6 and the dialyzer 3, it is possible to control the electromagnetic valves V1 through V6, the blood pump 4 and the dialysate supplying apparatus etc. by a control means (not shown) and to perform the charging step and the circulating step continuously and automatically.

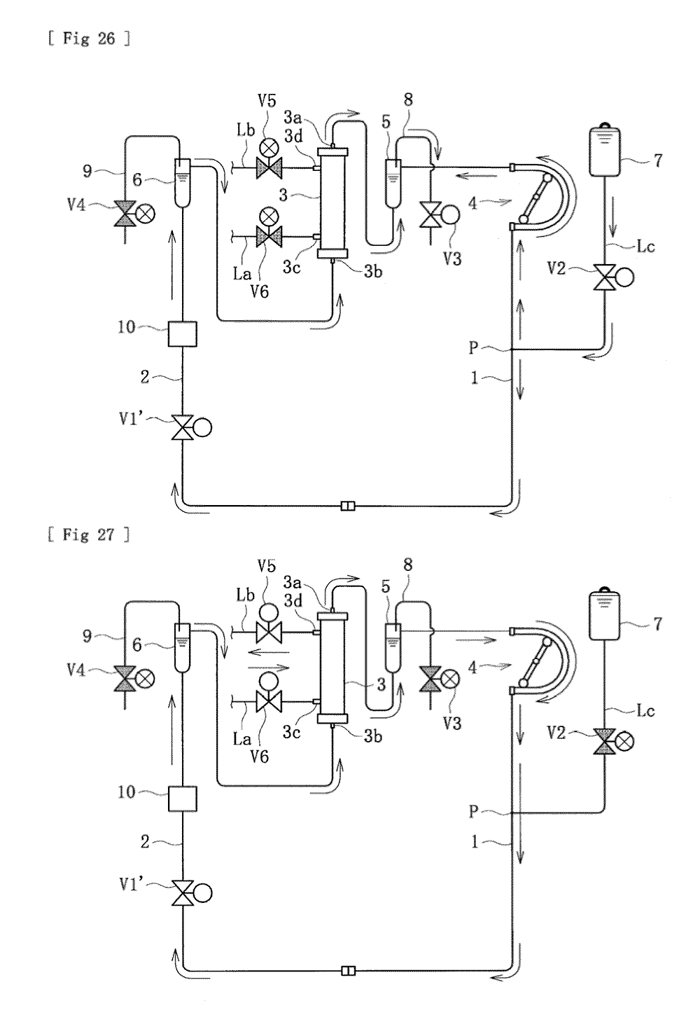

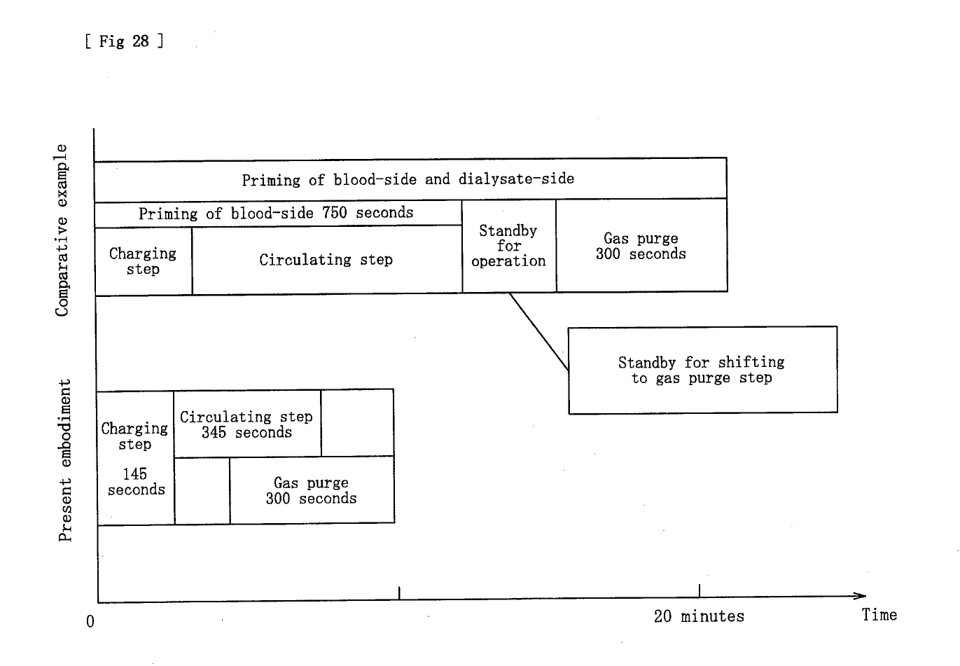

As shown in FIGS. 17 through 27, a system in which the first through fourth charging steps, the first and second circulating steps and the gas purge step are continuously performed is constructed on the basis of this embodiment. A comparative example is a so-called “automatic priming method” of the prior art. Comparison of the present embodiment and the comparative example will be depicted in FIG. 28.

A time required for priming is compared under same conditions using a container containing 1000 mL of saline, a dialyzer (wet type, and effective surface area: 1.5 m²) and blood circuits (general purpose articles). It was found that the present embodiment can reduce the time required for circulation because of improved air purge, and the time required for priming can be remarkably reduced by performing the gas purge step of the dialyzer 3 in parallel with the circulating step.

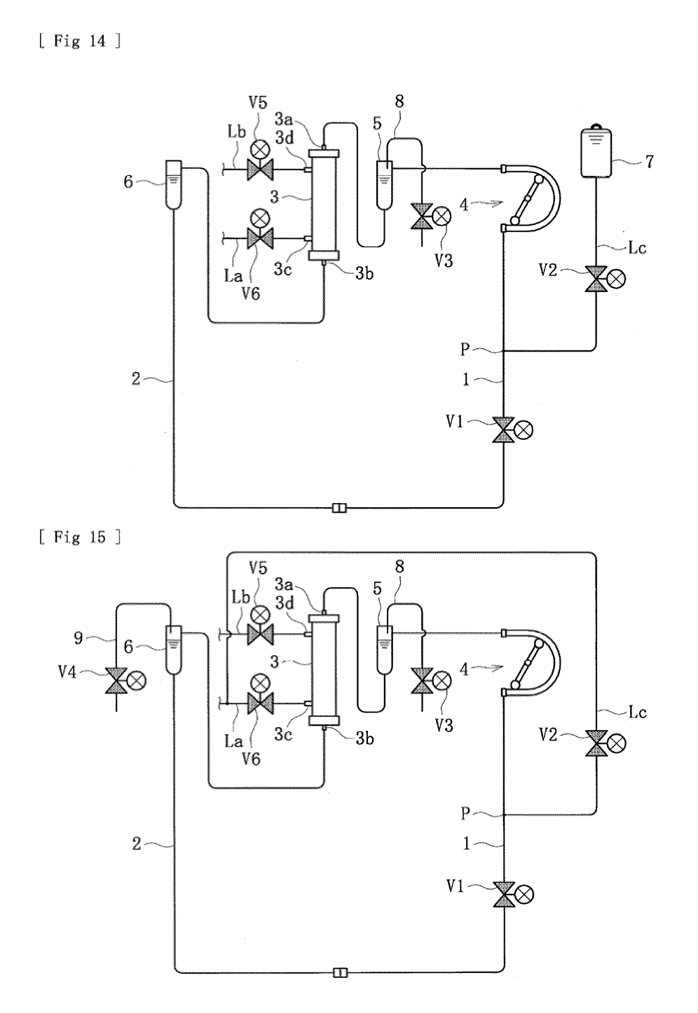

The present invention has been described with reference to the preferred embodiments. Obviously, modifications and alternations will occur to those of ordinary skill in the art upon reading and understanding the preceding detailed description. For example, it is possible to omit the preparatory priming step and to use the venous drip chamber 6 without overflow line as shown in FIG. 14. In addition it is preferable in an embodiment to omit the step shown in FIG. 3 and to drive the blood pump 4 so that the first priming step and the second priming step are simultaneously performed as shown in FIG. 4. In this case it is possible to perform the bubble purge in the dialyzer 3 in the priming as compared with an apparatus in which the first and second priming steps are successively performed.

In addition, although it is illustrated and described that the saline as the priming solution is supplied from the containing means 7 by gravity, it is possible to supply the saline using any pump arranged in the priming solution supplying line Lc. In addition as shown in FIG. 15, the dialysate as priming solution may be supplied to the blood circuits by extending the priming solution supplying line Lc from the dialysate introducing line La to the arterial blood circuit 1 (position between the connector “c” and the blood pump 4). In this case, it is preferable to interpose a filter in the priming solution supplying line Lc. Furthermore it may be possible to use other kind of priming solution (e.g. various kinds of electrolytic solutions).

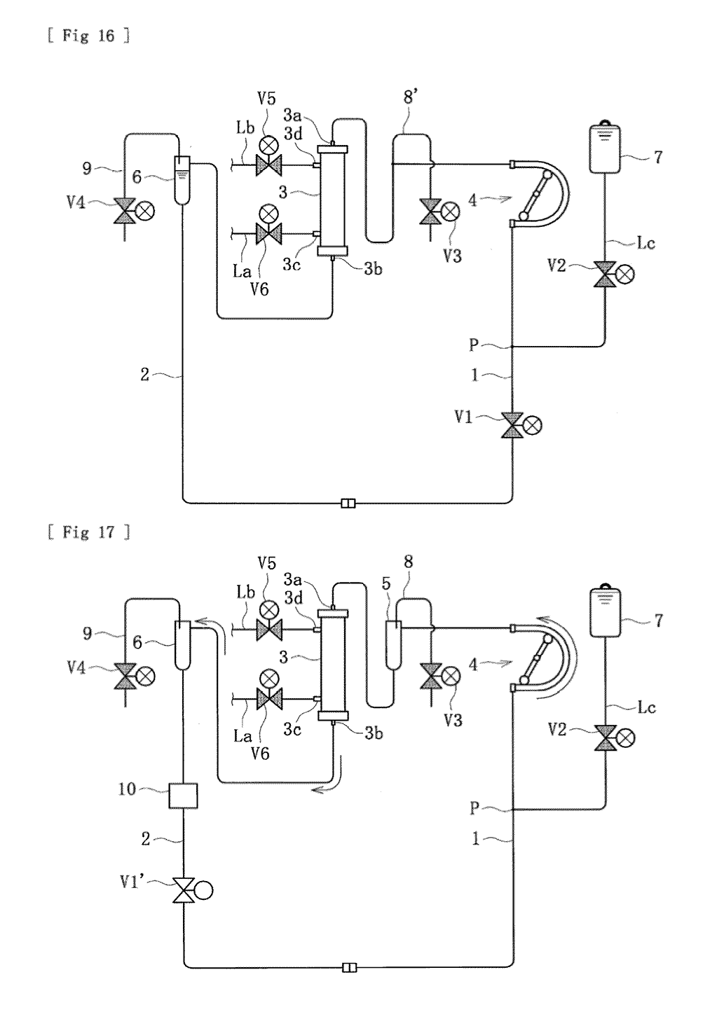

Although it is illustrated and described that the position for discharging the priming solution is selected in the arterial overflow line 8 extending from the arterial drip chamber 5 arranged intermediately of the arterial blood circuit 1 and in the venous overflow line 9 extending from the venous drip chamber 6, it is possible to select the position for discharging the priming solution at any position if is formed between the blood pump 4 of the arterial blood circuit 1 and the blood introducing port 3a of the dialyzer 3 and able to discharge the priming solution supplied from the priming solution supplying line Lc. For example as shown in FIG. 16, it is possible to omit the arterial drip chamber and to use a flow route 8′ branched from any position in the arterial blood circuit 1. In this case the flow route 8′ may be connected to the arterial blood circuit 1 via a “T” pipe, a “Y” pipe or a three-way branch pipe and may be connected to an access port such as a rubber button.

Although it is described that the valves V1 through V6 are electromagnetic valves, it is possible to use any types of valves if they can perform open/close operations. For example it is possible to omit the electromagnetic valve from the arterial overflow line 8 and to keep the arterial overflow line 8 always open and to manually close it using a forceps or a one-touch clamp. The use of a manually operated means in a place of less frequency of opening/closing operation enables to suppress total manufacturing cost of the blood purification apparatus. The present invention may be applied to other apparatus for extracorporeal blood purification of a patient (e.g. Hemodiafiltration, Hemofiltration, blood purification apparatus used in AFBF, plasma adsorption apparatus etc.) than the dialysis apparatus used in dialysis treatment.

The present invention can be applied to any other applications if they are a blood purification apparatus and its priming method in which the tip of the arterial blood circuit can be connected to and communicated with the tip of the venous blood circuit during priming of the blood purification apparatus before dialysis, and the blood purification apparatus can perform a priming solution charging step for supplying and charging the blood circuits with priming solution under a condition in which the tip of the arterial blood circuit and the tip of the venous blood circuit are connected to and communicated with each other; and a priming solution circulating step for forcing the charged priming solution to be flowed and circulated through the blood circuits by successively changing the driving speed of the blood pump after the priming solution charging step.

What is claimed is:

1. A blood purification apparatus comprising:
blood circuits including an arterial blood circuit and a venous blood circuit extracorporeally circulating blood of a patient from a tip of the arterial blood circuit to a tip of the venous blood circuit;
a blood purification device purifying blood of a patient flowing through the blood circuits interposed between the arterial blood circuit and the venous blood circuit and having a blood purification membrane and formed with a blood route through which blood of a patient flows and a dialysate route through which dialysate flows respectively through the blood purification membrane;
a blood pump arranged in the arterial blood circuit;
a dialysate input line and a dialysate discharging line respectively connected to an input port and a discharging port of the dialysate route of the blood purification device;
a blood introducing port formed on the blood purification device and adapted to be connected to the arterial blood circuit introducing blood into the blood route and a blood discharging port formed on the blood purification device and adapted to be connected to the venous blood circuit discharging blood from the blood route; and
a dialysate introducing port formed on the blood purification device and adapted to be connected to the dialysate introducing line introducing dialysate into the dialysate route and a dialysate discharging port formed on the blood purification device and adapted to be connected to the dialysate discharging line for discharging dialysate from the dialysate route;
wherein the tip of the arterial blood circuit removably connected to and communicated with the tip of the venous blood circuit during priming of the blood purification apparatus before dialysis characterized in that the blood purification apparatus can perform;
a priming solution charging step supplying and charging the blood circuits with priming solution under a condition in which the tip of the arterial blood circuit and the tip of the venous blood circuit are connected to and communicated with each other; and
a priming solution circulating step forcing the charged priming solution to be flowed and circulated through the blood circuits by successively changing the driving speed of the blood pump after the priming solution charging step.

2. The blood purification apparatus of claim 1 wherein the priming solution circulating step is performed by combining any two or more driving states of the blood pump of a normal rotation state, a reverse rotation state, and a stopped state.

3. The blood purification apparatus of claim 2 wherein at least two different driving speeds of the blood pump can be set in the normal rotation state and the reverse rotation state.

4. The blood purification apparatus of claim 1 further comprising:
an arterial drip chamber arranged intermediately of the arterial blood circuit;
an arterial overflow line extending from the arterial drip chamber and adapted to be opened and closed to communicate an air layer in the arterial drip chamber with the atmosphere;
a venous drip chamber arranged intermediately of the venous blood circuit;
a venous overflow line extending from the venous drip chamber and adapted to be opened and closed to communicate an air layer in the venous drip chamber with the atmosphere; and
a priming solution supplying line supplying the priming solution connected to the arterial blood circuit at a junction between the tip of the arterial blood line and the blood pump;
the priming solution charging step comprising:
a first charging step for normally rotating the blood pump by a discharging quantity corresponding to a volume of the flow route in the venous blood circuit from the blood discharging port of the blood purification device to the venous drip chamber;
a second charging step for discharging the priming solution from the venous overflow line after the stop of the blood pump by opening the venous overflow line and then forcing the priming solution from the priming solution supplying line to be flowed to the venous drip chamber of the venous blood circuit via the junction of the arterial blood circuit and the connection of the tip of the arterial blood circuit and the tip of the venous blood circuit;
a third charging step for discharging the priming solution from the arterial overflow line by closing the venous overflow line and opening the arterial overflow line and then forcing the priming solution from the priming solution supplying line to be flowed to the arterial drip chamber of the arterial blood circuit via the junction of the arterial blood circuit, the connection of the tip of the arterial blood circuit and the tip of the venous blood circuit, and the blood route of the blood purification device; and
a fourth charging step for discharging the priming solution from the arterial overflow line by normally rotating the blood pump and then forcing the priming solution to be flowed from the junction of the arterial blood circuit to the arterial drip chamber of the arterial blood circuit via the blood pump.

5. The blood purification apparatus of claim 4 wherein the priming solution circulating step comprises:
a first circulating step for circulating the charged priming solution in the blood circuits by closing the venous overflow line and the priming solution supplying line and then forcing the charged priming solution to be flowed; and
a second circulating step for purging air from the priming solution by opening the priming solution supplying line and opening either one of the arterial overflow line and the venous overflow line.

6. The blood purification apparatus of claim 4 wherein the priming solution charging step and the priming solution circulating step are performed by forcing the priming solution to be flowed from the bottom to the top of the blood purification device.

7. The blood purification apparatus of claim 6 wherein the priming solution charging step and the priming solution circulating step are performed by arranging the blood introducing port at the top of the blood purification device and the blood discharging port at the bottom of the blood purification device.

8. The blood purification apparatus of claim 4 wherein a bubble detector detecting bubbles in the blood circuits is arranged intermediately of one of the arterial blood circuit or the venous blood circuit, and wherein the air purging in the second circulating step is performed with interlocking with the detection of bubbles by the bubble detector.

9. The blood purification apparatus of claim 1 further comprising a gas purging step charging the dialysate route with dialysate by introducing the dialysate into the dialysate route of the blood purification device from the dialysate introducing line and discharging the dialysate from the dialysate discharging line, and wherein the gas purging step is automatically performed in at least one of the priming solution charging step and the priming solution circulating step.

10. A priming method for priming a blood purification apparatus comprising:
blood circuits including an arterial blood circuit and a venous blood circuit for extracorporeally circulating blood of a patient from a tip of the arterial blood circuit to a tip of the venous blood circuit;
a blood purification device for purifying blood of a patient flowing through the blood circuits interposed between the arterial blood circuit and the venous blood circuit of the blood circuits and having a blood purification membrane and formed with a blood route through which blood of a patient flows and a dialysate route through which dialysate flows respectively through the blood purification membrane;
a blood pump arranged in the arterial blood circuit;
a dialysate introducing line and a dialysate discharging line respectively connected to an introducing port and a discharging port of the dialysate route of the blood purification device;
a blood introducing port formed on the blood purification device and adapted to be connected to the arterial blood circuit for introducing blood into the blood route and a blood discharging port formed on the blood purification device and adapted to be connected to the venous blood circuit discharging blood from the blood route; and
a dialysate introducing port formed on the blood purification device and adapted to be connected to the dialysate introducing line introducing dialysate into the dialysate route and a dialysate discharging port formed on the blood purification device and adapted to be connected to the dialysate discharging line for discharging dialysate from the dialysate route;
the tip of the arterial blood circuit removably connected to and communicated with the tip of the venous blood circuit during priming of the blood purification apparatus before dialysis wherein the priming method of the blood purification apparatus comprises the steps of:
a priming solution charging step supplying and charging the blood circuits with priming solution under a condition in which the tip of the arterial blood circuit and the tip of the venous blood circuit are connected to and communicated with each other; and
a priming solution circulating step forcing the charged priming solution to be flowed and circulated through the blood circuits by successively changing the driving speed of the blood pump after the priming solution charging step.

11. The priming method for priming a blood purification apparatus of claim 10 wherein the priming solution circulating step further comprises the step of combining any two or more driving states of the blood pump of a normal rotation state, a reverse rotation state, and a stopped state.

12. The priming method for priming a blood purification apparatus of claim 11 wherein at least two different driving speeds of the blood pump can be set in the normal rotation state and the reverse rotation state.

13. The priming method for priming a blood purification apparatus of claim 10 wherein the blood purification apparatus further comprises:
an arterial drip chamber arranged intermediately of the arterial blood circuit;
an arterial overflow line extending from the arterial drip chamber and adapted to be opened and closed to communicate an air layer in the arterial drip chamber with the atmosphere;
a venous drip chamber arranged intermediately of the venous blood circuit;
a venous overflow line extending from the venous drip chamber and adapted to be opened and closed to communicate an air layer in the venous drip chamber with the atmosphere; and
a priming solution supplying line supplying the priming solution connected to the arterial blood circuit at a junction between the tip of the arterial blood line and the blood pump;
and wherein the priming solution charging step comprises the steps of:
a first charging step normally rotating the blood pump by a discharging quantity corresponding to a volume of the flow route in the venous blood circuit from the blood discharging port of the blood purification device to the venous drip chamber;
a second charging step discharging the priming solution from the venous overflow line after stopping the blood pump by opening the venous overflow line and then forcing the priming route from the priming solution supplying line to flow to the venous drip chamber of the venous blood circuit via the junction of the arterial blood circuit and the connection of the tip of the arterial blood circuit and the tip of the venous blood circuit;
a third charging step discharging the priming solution from the arterial overflow line by closing the venous overflow line and opening the arterial overflow line and then forcing the priming solution from the priming solution supplying line to flow to the arterial drip chamber of the arterial blood circuit via the junction of the arterial blood circuit, the connection of the tip of the arterial blood circuit and the tip of the vein-side blood circuit, and the blood route of the blood purification device; and
a fourth charging step discharging the priming solution from the arterial overflow line by normally rotating the blood pump and then forcing the priming solution to flow from the junction of the arterial blood circuit to the arterial drip chamber of the arterial blood circuit via the blood pump.

14. The priming method for priming a blood purification apparatus of claim 13 wherein the priming solution circulating step comprises the steps of:
a first circulating step circulating the charged priming solution in the blood circuits by closing the venous overflow line and the priming solution supplying line and then forcing the charged priming solution to be flow; and
a second circulating step purging air from the priming solution by opening the priming solution supplying line and opening one of the arterial overflow line and the venous overflow line.

15. The priming method for priming a blood purification apparatus of claim 13 wherein the priming solution charging step and the priming solution circulating step are performed by forcing the priming solution to flow from the bottom to the top of the blood purification device.

16. The priming method for priming a blood purification apparatus of claim 15 wherein the priming solution charging step and the priming solution circulating step are performed by arranging the blood introducing port at the top of the blood purification device and the blood discharging port at the bottom of the blood purification device.

17. The priming method for priming a blood purification apparatus of claim 13 wherein a bubble detector detecting bubbles in the blood circuits is arranged intermediately of the arterial blood circuit or the venous blood circuit, and wherein the air purging in the second circulating step is performed with interlocking with the detection of bubbles by the bubble detecting means.

18. The priming method for priming a blood purification apparatus of claim 10 further comprising the step of a gas purging step charging the dialysate route with dialysate by introducing the dialysate into the dialysate route of the blood purification device from the dialysate introducing line and discharging the dialysate from the dialysate discharging line, and wherein the gas purging step is automatically performed in at least one of the priming solution charging step and the priming solution circulating step.
